(19) United States
(12) Patent Application Publication
(10) Pub. No.: US 2025/0367436 A1
(43) Pub. Date: Dec. 4, 2025

(54) DEVICE AND METHOD FOR SLEEP APNEA TREATMENT

(71) Applicant: TorrentSpring LLC, Irvine, CA (US)

(72) Inventors: Jianlu Ma, Irvine, CA (US); Mengfan Li, Irvine, CA (US)

(73) Assignee: TorrentSpring LLC, Irvine, CA (US)

(21) Appl. No.: 19/221,271

(22) Filed: May 28, 2025

Related U.S. Application Data
(60) Provisional application No. 63/653,171, filed on May 29, 2024.

(51) Int. Cl. A61N 1/05; A61N 1/04; A61N 1/36

(52) U.S. Cl. CPC A61N 1/0548; A61N 1/0472; A61N 1/0551; A61N 1/36003

(57) ABSTRACT

A method for treating sleep apnea provides an implantable device having at least one layer of piezoelectric material sandwiched by an outer electrode and an inner electrode, implanting the implantable device inside the tongue of a patient, and generating electricity when the implantable device deforms, such that the electricity flows to the hyoglossus nerve to stimulate it, thereby moving the tongue to open the upper airway of the patient.

### BACKGROUND OF THE INVENTION

### 1. Field of the Invention

The present invention relates to treatment of sleep apnea, and in particular, to a devices and methods for treating sleep apnea.

### 2. Description of the Prior Art

Obstructive sleep apnea (OSA) is a common sleep disorder that disrupts normal breathing. With OSA, the air passages narrow as throat muscles relax during sleep, which causes a constant stopping and starting of breathing. One of the most common signs of sleep apnea is snoring.

When breathing is disrupted during sleep, one's body is deprived of oxygen which has negative health effects on many different parts of the body including the heart and brain. In addition to snoring, some OSA patients experience other symptoms like awakening during the night while gasping or choking, daytime sleepiness, high blood pressure, headaches, stroke, weight gain, depression or anxiety. Sleep apnea can also have a negative impact on a bed partner's sleep quality.

Causes of obstructive sleep apnea include high blood pressure, diabetes, smoking, obesity, narrowed airways or chronic nasal congestion.

In the United States, OSA affects an estimated 2% to 9% of the adult population, though experts believe there are a substantial number of undiagnosed cases. Living with OSA is associated with more serious health risks like stroke, heart disease, high blood pressure, and heart attack.

Both lifestyle and genetic factors can cause sleep apnea. While there is no cure, treatment can reduce the risk of heart and blood pressure-related problems as well as increase sleep quality.

Until recently, moderate to severe sleep apnea sufferers have had few treatment options, and they each came with significant challenges. Treatment with a CPAP machine has been the gold standard for treating sleep apnea, but it is not practical for many patients. Traditional surgical options that target the soft-palate tissue have only moderate success rates and often cause a great deal of pain and discomfort.

In recent years, several upper airway stimulation devices have been approved by the FDA, also known as hypoglossal nerve stimulation. These upper airway stimulation devices seem to make it possible for sleep apnea patients to maintain normal breathing patterns and finally be free from their CPAP machine. One of the FDA-approved implantable upper airway stimulation devices is the Inspire sleep apnea device. Inspire sleep apnea treatment opens the airway by moving the patient's tongue forward inside the mouth so that it does not block breathing passages. The device consists of three major components: a monitor that measures the breathing, a nerve stimulator that adjusts tongue placement, and a remote. The drawback is that this upper airway stimulation involves having a medical device surgically implanted under the skin, in the chest area, and some surgical incisions in other parts of the body under general anesthesia. This also makes the patient feel uncomfortable when they are awake.

Thus, there still remains a need for a new upper airway stimulation device and the method to treat the OSA.

### SUMMARY OF THE DISCLOSURE

To achieve the objectives of the present invention, the present invention provides a method for treating sleep apnea, which includes providing an implantable device having at least one layer of piezoelectric material sandwiched by an outer electrode and an inner electrode, implanting the implantable device inside the tongue of a patient, and generating electricity when the implantable device deforms, such that the electricity flows to the hyoglossus nerve to stimulate it, thereby moving the tongue to open the upper airway of the patient.

### BRIEF DESCRIPTION OF THE DRAWINGS

FIG. 1 shows the normal upper airway for a human being.

FIG. 2 shows the blocked upper airway (sleep apnea) due to the blockage in upper airway caused by the back movement of the tongue and/or the abnormal placement of the soft palate.

FIG. 3 shows an implanted device according to the present invention, implanted in the tongue, in a deformation-free configuration when the upper airway is open.

FIG. 4 shows the implanted device of FIG. 3 deformed when the upper airway is blocked and prior to the upper airway being unblocked.

FIG. 5 shows the implanted device of FIG. 3 implanted at a different orientation in a deformation-free configuration when the upper airway is open.

FIG. 6 shows the implanted device of FIG. 5 deformed when the upper airway is blocked and prior to the upper airway being unblocked.

FIG. 7 shows the implanted device of FIG. 3, implanted in soft palate, in a deformation-free configuration when the upper airway is open.

FIG. 8 shows the implanted device of FIG. 7 deformed when the upper airway is blocked and prior to the upper airway being unblocked.

FIG. 9 is a schematic view of a first embodiment of an implanted device according to the present invention.

FIG. 10 is a schematic view of a second embodiment of an implanted device according to the present invention.

FIG. 11 is a schematic view of a third embodiment of an implanted device according to the present invention.

FIG. 12A shows how the implanted device in FIG. 7 operates in a deformation-free configuration when the upper airway is open.

FIG. 12B shows how the implanted device in FIG. 8 operates when deformed when the upper airway is blocked and prior to the upper airway being unblocked.

FIG. 13A shows the implanted device of FIG. 3 implanted on the surface of the tongue, and how it operates in a deformation-free configuration when the upper airway is open.

FIG. 13B shows how the implanted device in FIG. 13A operates when deformed when the upper airway is blocked and prior to the upper airway being unblocked.

FIG. 14A shows how the implanted device in FIG. 5 operates in a deformation-free configuration when the upper airway is open.

FIG. 14B shows how the implanted device in FIG. 6 operates when deformed when the upper airway is blocked and prior to the upper airway being unblocked.

FIG. 15 is a parallel circuit structure of a double-layer device with piezoelectric material.

FIG. 16 is a series circuit structure of a double layer device with piezoelectric material.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The present invention provides a medical device that is implanted in a patient's soft-palate or tongue. The implanted device includes piezoelectric elements. Once implanted, the device will be deformed (bent or stretched or compressed) by the movement of the soft-palate (vibration or other abnormal movement) or the tongue (choking, or backward movement during sleep) when the patient snores or chokes (i.e., soft-palate or tongue are in abnormal placement or move abnormally). The deformation on the implanted devices with piezoelectric element(s) can generate surface charge or voltage. The generated surface charge or voltage can send a pulse to the airway-patency-related nerves (such as hypoglossal nerve) or muscles that control the soft-palate or tongue's motor function (stimulation). The stimulation causes the tongue or soft palate to move back to their normal position in the mouth, clearing up more space for air to pass through. Thus, the present invention provides a device with piezoelectric element(s) can stimulate the hypoglossal nerve and airway muscles to ensure normal breathing.

The implanted device with piezoelectric element(s) can react to and monitor the abnormal movement or placement of the soft-palate and tongue and delivers stimulation to maintain the soft-palate or tongue out of the airway.

Piezoelectric material are the materials that have piezoelectricity. Piezoelectricity is the generation of electric charges/voltage due to the effect of mechanical strain/deformation/stress. Mechanical strain/deformation/stress on piezoelectric materials will produce a polarity/potential in the material with each deformation movement. When pressure or deformation is applied to a piezoelectric material, a dipole and net polarization/potential are produced in the direction of the applied stress/deformation. Piezoelectricity converts mechanical energy into electricity. In the present invention, the conversion between mechanical energy/deformation/stress to electricity/potential is used.

The piezoelectric materials can be either piezoelectric ceramic and some ferroelectric materials (for example, BaTiO3, LiTaO3, PZT, PbTiO3, etc.), or piezoelectric polymer materials (for example, PVDF, and the PVDF copolymers, VF2VF3, etc.), or the combination of the different types of piezoelectric materials such as WS2 or MoS2. In addition, the electret materials like fluoropolymers, synthetic polymers such as PTFE (polytetrafluoroethylene), polypropylene such as polypropylene or polyethyleneterephthalate (PET), could be used to generate the electric voltage/pulse as well. The piezoelectric/electret materials can be in the form of layers, beams, sheet, tubular, thin film, surface layer, deposited or plated layer, printed layer, powder form, fibers, wires, strip form, electrospun fibers and electrospun nanofibers etc.

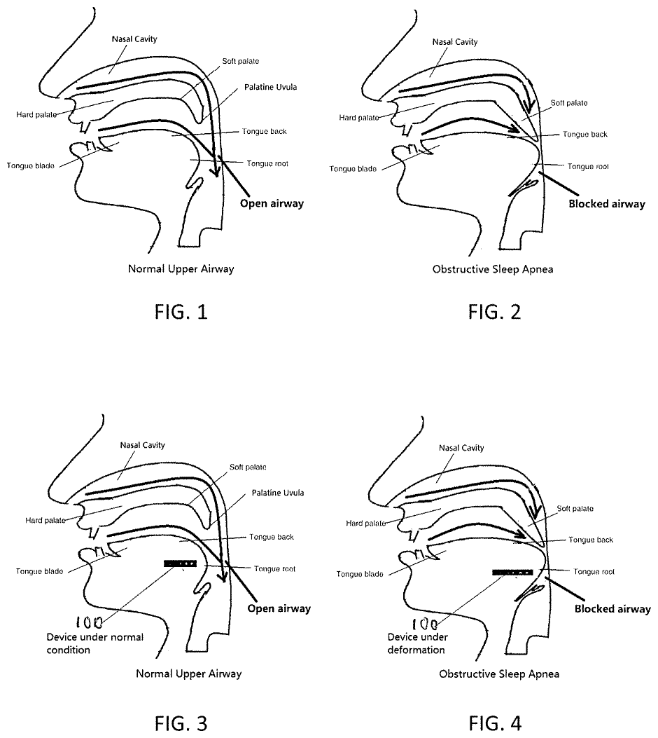

FIG. 1 shows the normal upper airway for a human being when it is opened for breathing. FIG. 2 shows the blocked upper airway (sleep apnea) due to the blockage in the upper airway caused by the back movement of the tongue and/or the abnormal placement of the soft palate.

Obstructive Sleep Apnea (OSA) is a prevalent condition characterized by repeated episodes of partial or complete obstruction of the upper airway during sleep, leading to disrupted sleep and decreased oxygen saturation. The primary anatomical cause of OSA is the relaxation and posterior displacement of the tongue muscles, which can obstruct the airway. The hyoglossus nerve, responsible for controlling tongue movement, offers a promising target for therapeutic intervention. The present invention leverages the principles of piezoelectricity to address this issue. Piezoelectric materials have the unique ability to generate an electric charge in response to mechanical stress. In one embodiment of the implanted device of the present invention, a strategically positioned piezoelectric element is implanted within the tongue at a location that undergoes maximum deformation when the tongue muscles relax. See FIGS. 3-6. As the tongue moves backward during sleep, it deforms the piezoelectric element, generating an electrical charge. This generated electricity is then used to stimulate the hyoglossus nerve, causing it to contract and reposition the tongue forward, thus maintaining an open airway. This self-contained system eliminates the need for external power sources, enhancing patient comfort and compliance. By harnessing the body's natural movements to generate the necessary stimulation, the implanted device provides a novel, minimally invasive solution to effectively manage OSA, improving patient outcomes and quality of life.

FIG. 3 shows an implanted device 100 according to the present invention, implanted in the tongue, in a deformation-free configuration when the upper airway is open.

FIG. 4 shows the implanted device 100 of FIG. 3 deformed when the upper airway is blocked and prior to the upper airway being unblocked. The deformation of the implanted device 100 will cause the upper airway to be unblocked when its deformation will move the tongue to the orientation shown in FIG. 3.

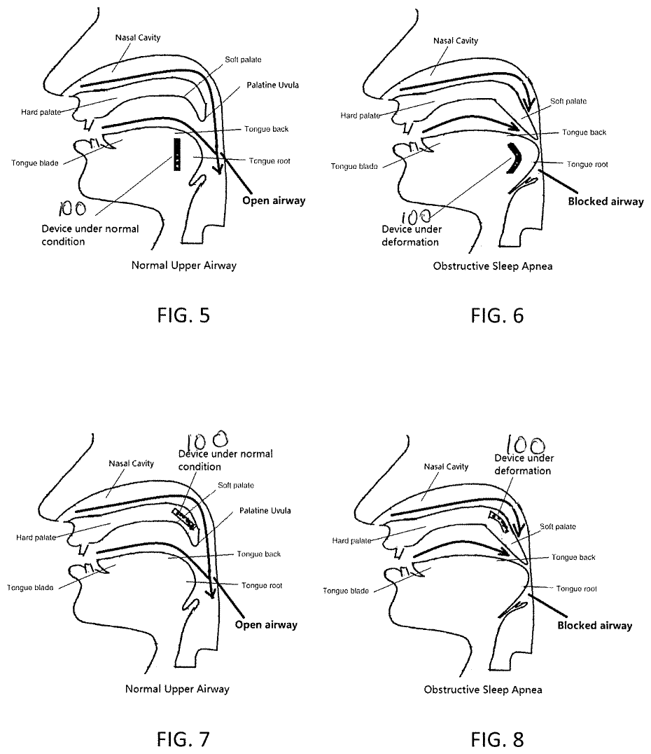

FIG. 5 shows the implanted device 100 of FIG. 3 implanted at a different orientation inside the tongue in a deformation-free configuration when the upper airway is open.

FIG. 6 shows the implanted device 100 of FIG. 5 deformed when the upper airway is blocked and prior to the upper airway being unblocked. The deformation of the implanted device 100 will cause the upper airway to be unblocked when its deformation will move the tongue to the orientation shown in FIG. 5.

FIG. 7 shows the implanted device 100 of FIG. 3, implanted in soft palate, in a deformation-free configuration when the upper airway is open.

FIG. 8 shows the implanted device of FIG. 7 deformed when the upper airway is blocked and prior to the upper airway being unblocked. The deformation of the implanted device 100 will cause the upper airway to be unblocked when its deformation will move the soft palate back to the orientation shown in FIG. 7.

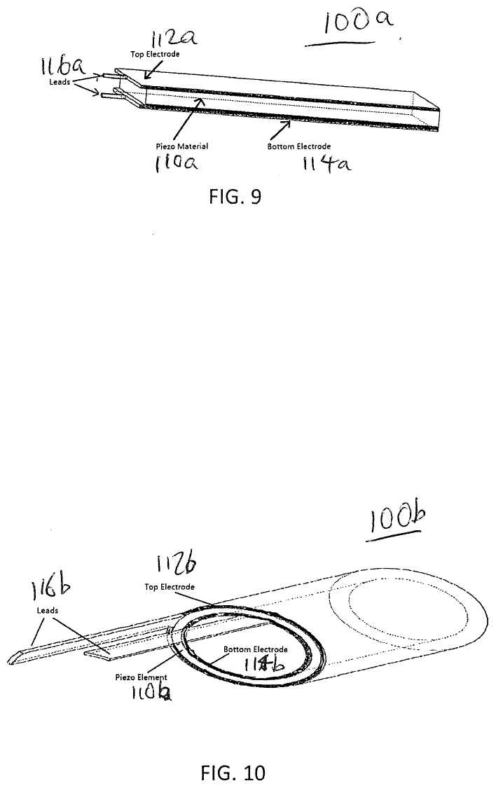

FIG. 9 is a schematic view of a first embodiment of an implanted device 100a according to the present invention. The implanted device 100a has a layer of piezoelectric material 110a sandwiched between a top electrode 112a and a bottom electrode 114a. Leads 116a can extend from the top electrode 112a and the bottom electrode 114a.

FIG. 10 is a schematic view of a second embodiment of an implanted device 100b according to the present invention. The implanted device 100b has a layer of piezoelectric material 110b sandwiched between an outer electrode 112b and an inner electrode 114b. Leads 116b can extend from the outer electrode 112b and the inner electrode 114b. The difference between the implanted devices 100a and 100b is that the implanted device 100a has a planar configuration, while the implanted device 100b has a tubular or concentric configuration.

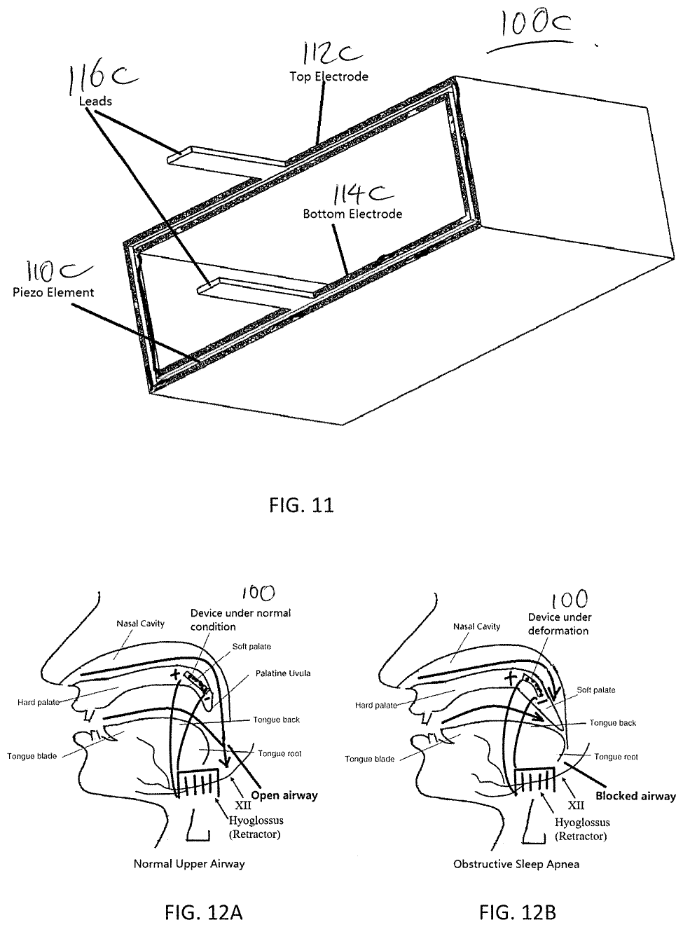

FIG. 11 is a schematic view of a third embodiment of an implanted device according to the present invention. The implanted device 100c has a layer of piezoelectric material 110c sandwiched between an outer electrode 112c and an inner electrode 114c. Leads 116b can extend from the outer electrode 112c and the inner electrode 114c. The difference between the implanted devices 100b and 100c is that the implanted device 100c has a four-sided configuration, while the implanted device 100b has a tubular or concentric configuration.

FIGS. 12A and 12B correspond to FIGS. 7 and 8 except that they show the electrical connections. The hyoglossus muscle is one of the four intrinsic muscles of the tongue. It is a quadrilateral muscle that originates along the whole length of the hyoid bone and inserts into the side of the tongue. The hyoglossus acts to both depress and retract the tongue. It receives its motor innervation via the hypoglossal nerve. When soft palate is relaxed (i.e., moves downward), the deformation on the implanted device 100 can generate electricity, the electricity can flow through the leads 116 to the hyoglossus nerve to stimulate the nerve, thereby causing the tongue to contract to move back to the normal position. This location for implanting the device 100 works in the OSA patient population that involves the soft palate.

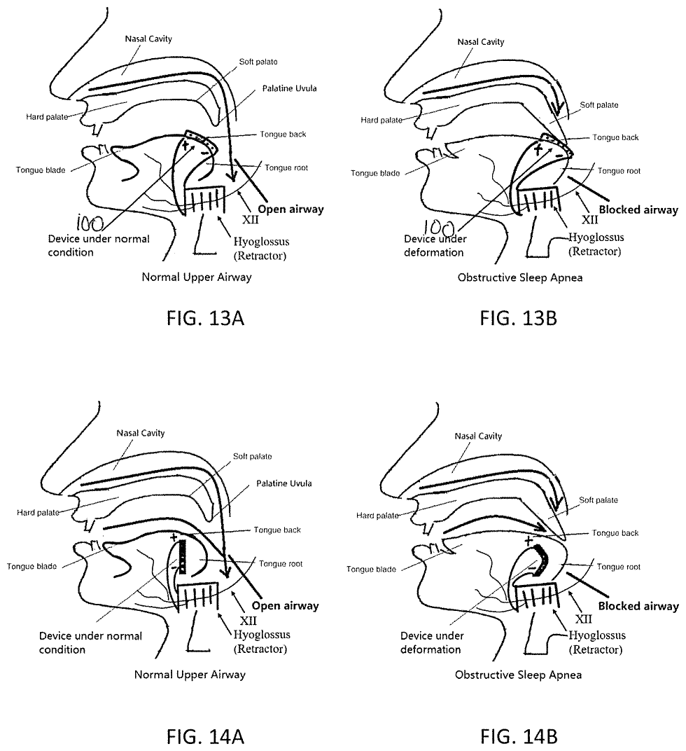

FIGS. 13A and 13B show the device 100 attached on the surface of the tongue near the back of the tongue. The deformation is applied to the device 100 when the tongue muscles are relaxed, and tongue moves backward. The deformation leads to the generation of electricity and the electricity flows though the leads 116 that are connected to the hyoglossus nerve to stimulate it, so that it can pull the tongue muscles back to the normal position, thereby leaving the upper airway open.

FIGS. 14A and 14B correspond to FIGS. 5 and 6 except that they show the electrical connections. The device 100 is implanted in the tongue (near the back of the tongue where the deformation is maximum when tongue muscle is relaxed), and the deformation is applied to the device 100 when the tongue muscles are relaxed, and tongue moves backward. The deformation leads to the generation of electricity and the electricity flows though the leads 116 that are connected to the hyoglossus nerve to stimulate it, so that it can pull the tongue muscles back to the normal position, thereby leaving the upper airway open.

The device 100 can either have lead(s) 116, or be leadless. Instead of top/outer and bottom/inner electrodes, the surface of the piezoelectric element can be coated or sputtered, or deposited with some conductive materials or adhesives or some other dielectric materials, so that no lead is needed to create the electric pulse to stimulate the nerve and/or the muscles that control the upper airway passageway.

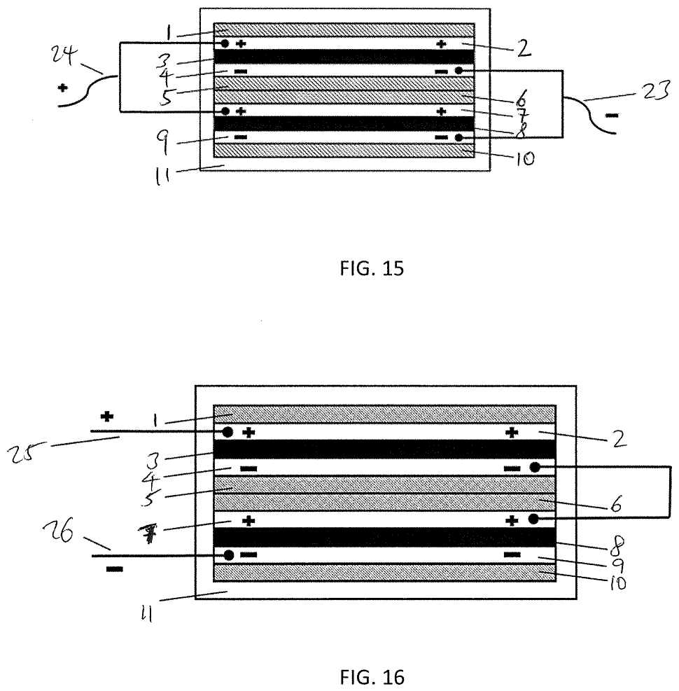

For example, consider FIGS. 15 and 16. FIG. 15 is a parallel circuit structure of a double-layer device with piezoelectric material. FIG. 16 is a series circuit structure of a double layer device with piezoelectric material. The structures shown in FIGS. 15 and 16 can be used for any of the devices 100a, 100b, 100c shown in FIGS. 9-11. FIGS. 9-11 are simplified conceptual diagrams for the devices 100a, 100b and 100c, while FIGS. 15 and 16 illustrate more detailed structure that can be used for the devices 100a, 100b, 100c.

Referring to FIGS. 15 and 16, the layers 3 and 8 represent the layers of piezoelectric material. These materials are generally piezoelectric materials or piezoelectric material compounds. Piezoelectric materials can be made from some relatively soft composite materials (such as PVDF/ZnO, BaTiO3, etc./Ag, carbon-based materials, metal oxides, etc.), and the general composite materials contain three materials: the first material is PVDF and its polymer, which is the main material that produces piezoelectric properties; the second material is generally a material that enhances the β phase content of PVDF and its polymer and also has piezoelectric properties, such as zinc oxide, BaTiO3, etc.; and the third material is conductive materials, which aim to reduce internal resistance, improve material flexibility, enhance charge flow, etc., such as Ag, carbon-based materials, metal oxides, etc. The main forms of production are electrospinning, stretching, heat treatment, polarization, etc., and the form is generally softer, and it is biocompatible and can be implanted into the back of the tongue, which can resist saliva, and normal saline. Due to the limited piezoelectric properties of single-layer piezoelectric materials, the present invention uses two or more layers of piezoelectric materials to achieve this performance, and at the same time, the charge output can be enhanced with the help of friction force. The multi-layer piezoelectric materials are connected in series or parallel, and their efficiency is more than 95% compared with that of a single layer. The electricity needed can be generated either by the pure formation of the piezoelectric elements, or by the friction action among the layers. Usually, the electricity generated by the friction is much higher than that from deformation.

The layers 2, 4, 7, 9 in FIGS. 15 and 16 are electrode layers, and generally metal electrodes, that are made from Ag, Au, Pt, Al, and similar materials, such as graphene, carbon nanotubes, etc. The bonding method among different layers includes glue bonding (generally aluminum, copper, etc.), spraying (generally conductive silver glue, etc.), sputtering (generally platinum, gold, etc.), and screen printing, among others.

Layers 1, 5, 6, 10 in FIGS. 15 and 16 are insulation materials, generally polyimide (PI), and PMMA (polymethyl methacrylate), among others.

Layer 11 in FIGS. 15 and 16 represent the second protective layer, and the material is soft and provides insulation as an isolation protective layer, and can be made of PDMS.

Leads 23, 24, 25 and 26 are also shown in FIGS. 15 and 16. The leads 24 and 25 are wire positive junctions, connecting cuff positives, while leads 23 and 26 are wire negative junctions, connecting cuff positives. The cuff is the component in this device 100 which connects with the nerve under the tongue directly to provide stimulation.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A method for treating sleep apnea, comprising the steps of:
providing an implantable device having at least one layer of piezoelectric material sandwiched by an outer electrode and an inner electrode;
implanting the implantable device inside the tongue of a patient; and
generating electricity when the implantable device deforms, such that the electricity flows to the hyoglossus nerve to stimulate it, pulling the tongue muscles to open the upper airway of the patient.

2. The method of claim 1, wherein the implantable device further includes at least one lead connected to one of the electrodes and the hyoglossus nerve.

3. The method of claim 1, wherein the implantable device further includes an insulation layer surrounding the outer electrode.

4. A method for treating sleep apnea, comprising the steps of:
providing an implantable device having at least one layer of piezoelectric material sandwiched by an outer electrode and an inner electrode;
implanting the implantable device inside the soft palate of a patient; and
generating electricity when the implantable device deforms, such that the electricity flows to the hyoglossus nerve to stimulate it, thereby causing the tongue to contract to move back to open the upper airway of the patient.

5. The method of claim 4, wherein the implantable device further includes at least one lead connected to one of the electrodes and the hyoglossus nerve.

6. The method of claim 4, wherein the implantable device further includes an insulation layer surrounding the outer electrode.
